(19) United States
(12) Patent Application Publication
(10) Pub. No.: US 2022/0026083 A1
(43) Pub. Date: Jan. 27, 2022

(54) AIR CONDITIONING DEVICE

(71) Applicant: MediFiber Inc., Gyeonggi-do (KR)

(72) Inventors: Joonggu WOO, Seoul (KR); Hyunho LEE, lncheon (KR); Yongmin KIM, Gyeonggi-do (KR)

(21) Appl. No.: 17/120,086

(22) Filed: Dec. 11, 2020

(30) Foreign Application Priority Data
Jul. 27, 2020 (KR) 10-2020-0093313

(51) Int. Cl. F24F 3/16; F24F 13/28; F24F 8/175; F24F 8/20; D06M 11/83; D06M 16/00

(52) U.S. Cl. CPC F24F 3/16; F24F 13/28; F24F 8/175; B82Y 5/00; D06M 11/83; D06M 16/00; F24F 8/20

(57) ABSTRACT

The present invention relates to an air conditioning device. The air conditioning device sucks air through a suction portion and sending the sucked air to a blowing portion to perform air conditioning, wherein the suction portion includes a virus killing portion formed of a fiber to which copper ions are bonded and killing a virus, the virus killing portion includes a caz fabric configured to contain 5 to 50 wt % of an antiviral alginic acid conjugate fiber configured so that the copper ions are contained in an amount of 6,000 to 100,000 ppm in a fiber, and the virus killing portion has a differential pressure exceeding 0 Pa and less than 10 Pa.

### CROSS-REFERENCE TO RELATED APPLICATION(S)

This application claims benefit of priority to Korean Patent Application No. 10-2020-0093313 filed on Jul. 27, 2020 in the Korean Intellectual Property Office, the disclosure of which is incorporated herein by reference in its entirety.

### BACKGROUND

### 1. Field

The present invention relates to an air conditioning device, and more particularly, to an air conditioning device that performs not only a function of blocking a virus but also a function of killing the virus by including both of a virus blocking portion and a virus destroying portion.

### 2. Description of Related Art

A case where modern people are living in enclosed indoor spaces such as homes, schools, and offices has increased, and as a result of an effort to maintain these enclosed indoor spaces in a pleasant environment, the spread of air conditioning devices in various buildings has increased since a long time ago.

Generally, the air conditioning device is a device for creating a pleasant indoor environment by simultaneously controlling indoor temperature, humidity, and cleanliness.

The air conditioning devices installed in the various buildings not only perform main functions such as cooling, heating, and air cleaning according to their respective purposes, but also perform a function of removing various floating dusts or the like in air introduced into an interior through a duct by a net filter mounted on an air inlet port side.

However, the net filter used in such an air conditioning device only serves to filter the dusts included in the air introduced into an air inlet port so as not to be introduced into a body, but has no special function. Therefore, when air including a bacterium or a virus passes through the air conditioning device and is then released into the interior as it is, there was a problem that the air containing the bacterium or the virus contaminates walls, furniture, clothing, or the like, in the interior and in particular, damages user's health.

In particular, there is a problem that the transmission and the spread of the virus may occur through air conditioning device in a situation where COVID-19 is currently breaking out worldwide.

Therefore, research and development of an air conditioning device capable of preventing the transmission and the spread of the virus are currently being actively conducted in the field of the air conditioning device.

### SUMMARY

The present invention has been made in an effort to solve the problems of the related art as described above, and an object of the present invention is to an air conditioning device that performs not only a function of blocking a virus but also a function of killing the virus.

An air conditioning device according to an embodiment of the present invention is an air conditioning device that sucks air through a suction portion and sending the sucked air to a blowing portion to perform air conditioning, wherein the suction portion includes a virus killing portion formed of a fiber to which copper ions are bonded and killing a virus, the virus killing portion includes a caz fabric configured to contain 5 to 50 wt % of an antiviral alginic acid conjugate fiber configured so that the copper ions are contained in an amount of 6,000 to 100,000 ppm in a fiber, and the virus killing portion has a differential pressure exceeding 0 Pa and less than 10 Pa.

The caz fabric may be configured by producing the antiviral alginic acid conjugate fiber by mixing 5 to 10 wt % of sodium alginate polymer, 1 to 3 wt % of copper sulfate powder or copper chloride powder, and distilled water as a balance with each other and stirring them at 40 to 60° C. to produce a copper sulfate-alginate spinning solution, degassing the copper sulfate-alginate spinning solution in order to remove air bubbles, filtering the degassed copper sulfate-alginate spinning solution, quantitatively discharging the filtered copper sulfate-alginate spinning solution through a gear pump, coagulating the discharged copper sulfate-alginate spinning solution in a coagulation solution containing calcium chloride, sequentially passing the coagulation through a water tank and an oil tank, drying the coagulation, and then winding the coagulation.

The suction portion may further include a virus blocking portion formed in a membrane form to block the virus or a droplet containing the virus, and the virus killing portion may be configured to primarily destroy the viruses passing through the virus killing portion, and be located in front of the virus blocking portion so as to secondarily eradicate residual viruses collected on a surface of the virus blocking portion by the virus blocking portion when the virus blocking portion blocks the viruses from passing therethrough, such that the viruses are collected on the surface of the virus blocking portion.

The virus killing portion may be formed of a fiber having hydrophilicity, and the virus blocking portion may be formed of a membrane filter having hydrophobicity.

The virus blocking portion may have a form of a nano-membrane filter, and the virus killing portion may be formed by spinning a nano-fiber forming the nano-membrane filter of the virus blocking portion on a fabric and then overlapping the caz fabric with the nano-fiber to be configured to be located in front of the virus blocking portion.

The fabric may be a smooth nonwoven fabric.

The virus blocking portion may have a form of a nano-membrane filter, and the virus killing portion may be formed by spinning a nano-fiber forming the nano-membrane filter of the virus blocking portion on the caz fabric in an electro-spinning manner to be configured to be located in front of the virus blocking portion.

The suction portion may further include a filter portion filtering foreign materials sucked in the air sucked through the suction portion, and the suction portion may be formed so that the virus killing portion is formed at the forefront of the suction portion and the virus blocking portion and the filter portion are sequentially toward a rear.

The air conditioning device may be at least one of an air conditioner for an interior, an air conditioner for a vehicle, an air cleaner, an electric fan, or a heater.

The air conditioning device according to an embodiment of the present invention includes the virus killing portion disposed in front of the suction portion and having a differential pressure exceeding 0 Pa and less than 10 Pa to be capable of killing the virus in sucked air without substantially hindering air suction of the air conditioning device.

In addition, the air conditioning device according to an embodiment of the present invention may include both of the virus killing portion and the virus blocking portion to perform not only a function of blocking the virus but also a function of killing the virus, thereby maximizing safety of a user and efficiently preventing the spread of the virus.

In addition, in the air conditioning device according to an embodiment of the present invention, the virus killing portion is configured to be located in front of the virus blocking portion, such that the viruses are primarily destroyed while passing through the virus killing portion, and residual viruses that are not destroyed by the virus killing portion and pass through the virus killing portion are blocked by the virus blocking portion to be collected on a surface of the virus blocking portion and are again secondarily destroyed by the virus killing portion. As a result, an ability to kill the virus may be significantly improved.

In addition, in the air conditioning device according to an embodiment of the present invention, the virus killing portion is produced using the caz fabric, such that an antibacterial ability is improved as compared with an existing antibacterial fiber.

### BRIEF DESCRIPTION OF DRAWINGS

FIG. 1 is a perspective view of an air conditioning device according to an embodiment of the invention.

FIG. 2 is a conceptual diagram of stacking of a filter portion of the air conditioning device according to an embodiment of the present invention.

FIGS. 3A and 3B are comparison conceptual diagrams of a virus blocking portion (membrane filter) of the air conditioning device according to an embodiment of the present invention and a charged melt blown filter according to the related art.

FIG. 4 is a result table illustrating experimental results for an antimicrobial property of a virus killing portion of the air conditioning device according to an embodiment of the present invention.

FIG. 5 is a bar graph comparison table illustrating an evaluation of inactivation of an indicator virus (MS-2 bacteriophage) of the virus killing portion of the air conditioning device according to an embodiment of the present invention.

### DETAILED DESCRIPTION

Objects, specific advantages, and novel features of the present invention will become more apparent from the following detailed description and preferred embodiments associated with the accompanying drawings. In the present specification, in adding reference numerals to components of respective drawings, it should be noted that the same components will be denoted by the same reference numerals as possible even though they are indicated on different drawings. In addition, in describing the present invention, when it is determined that a detailed description of the known art related to the present invention may unnecessarily obscure the gist of the present invention, the detailed description thereof will be omitted.

Hereinafter, the present invention will be described in detail together with the drawings illustrated below.

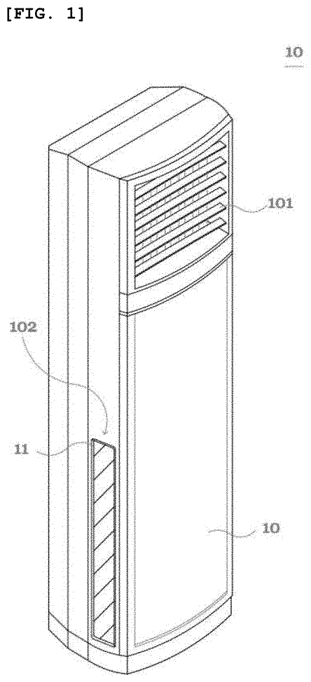

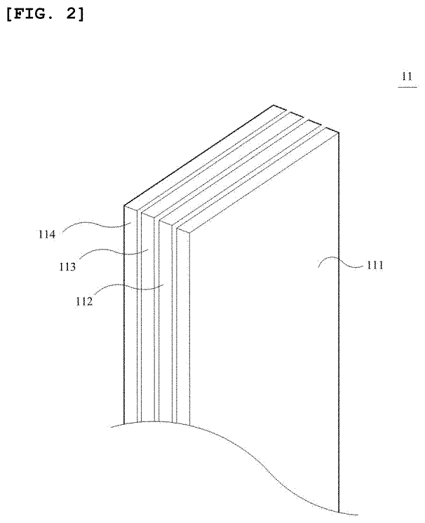

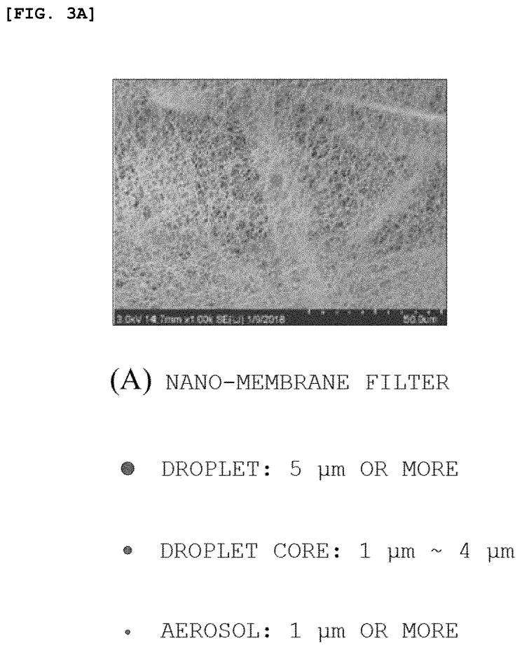

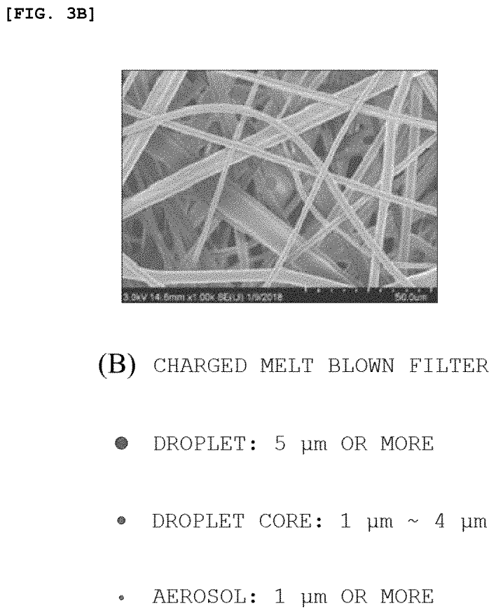

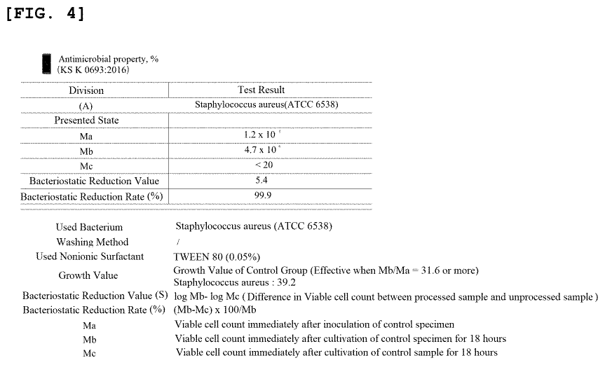

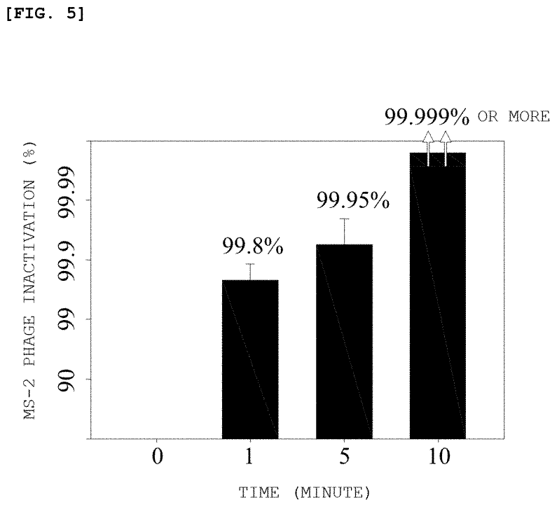

FIG. 1 is a perspective view of an air conditioning device according to an embodiment of the invention, FIG. 2 is a conceptual diagram of stacking of a filter portion of the air conditioning device according to an embodiment of the present invention, FIGS. 3A and 3B are comparison conceptual diagrams of a virus blocking portion (membrane filter) of the air conditioning device according to an embodiment of the present invention and a charged melt blown filter according to the related art, FIG. 4 is a result table illustrating experimental results for an antimicrobial property of a virus killing portion of the air conditioning device according to an embodiment of the present invention, and FIG. 5 is a bar graph comparison table illustrating an evaluation of inactivation of an indicator virus (MS-2 bacteriophage) of the virus killing portion of the air conditioning device according to an embodiment of the present invention.

As illustrated in FIGS. 1 to 5, an air conditioning device 1 according to an embodiment of the present invention includes an air conditioning device body portion 10 and a filter portion 11. Here, the air conditioning device 1 may correspond to an air conditioner for an interior, an air conditioner for a vehicle, an air cleaner, an electric fan, a heater, or the like. In addition, the air conditioning device 1 is not limited in terms of a size, and may correspond to a large air conditioning device installed in various buildings or a small air conditioner installed in an interior such as a home.

A configuration of the air conditioning device 1 illustrated in the present invention is an example of one of the devices described above for convenience of explanation, and a core configuration of the air conditioning device 1 corresponds to a suction portion 102 in which the filter portion 11 is disposed. Therefore, it should be noted that when a type of the air conditioner 1 is applied to each of the air conditioner for an interior, the air conditioner for a vehicle, the air cleaner, the electric fan, and the heater described above, configurations other than the core configuration may be easily changed by those skilled in the art.

Hereinafter, the air conditioning device 1 according to the present invention will be described in detail.

The air conditioning device body portion 10 includes a blowing portion 101 and the suction portion 102, and may suck air through the suction portion 102, simultaneously control indoor temperature, humidity, cleanliness, and the like, through known devices formed inside the air conditioner body portion 10, and then discharge the controlled air through the suction portion 102. Therefore, the air conditioning device 1 may create a pleasant indoor environment.

The blowing portion 101 may be formed at an upper side of the air conditioning device body portion 10, and discharge the air sucked from the suction portion 102 again. The blowing portion 101 may discharge the air after being processed inside the air conditioning device body portion 10 so that an air condition satisfies a preset condition, to the outside.

The suction portion 102 may be formed at a lower side of the air conditioning device body portion 10 and suck external air.

Only a virus killing portion 111 may be formed on an outer side of the suction portion 102, and the air sucked into the suction portion 102 may pass through the virus killing portion 111 and be introduced then into the air conditioning device body portion 10.

The virus killing portion 111 is formed of a fiber to which copper ions are bonded, and kills a virus. In this case, the virus killing portion 111 may have a differential pressure exceeding 0 Pa and less than 10 Pa, and preferably exceeding than 0 Pa and less than 7 Pa.

Therefore, the air conditioning device 1 according to the present invention includes the virus killing portion 111 disposed in front of the suction portion 102 and having the differential pressure exceeding 0 Pa and less than 10 Pa to be capable of killing the virus in the sucked air without substantially hindering air suction of the air conditioning device 1.

The virus killing portion 111 may be configured to primarily destroy the viruses passing through the virus killing portion 111 and be located in front of a virus blocking portion 112 to be described below so as to secondarily eradicate residual viruses collected on a surface of the virus blocking portion 112 by the virus blocking portion 112 when the virus blocking portion 112 blocks the viruses from passing therethrough, such that the viruses are collected on the surface of the virus blocking portion 112.

The virus killing portion 111 may include a caz fabric, which is a copper ion-binding polymer fiber configured to contain 5 to 50 wt % of an antiviral alginic acid conjugate fiber configured so that the copper ions are contained in an amount of 6,000 to 100,000 ppm in a fiber.

The caz fabric may be configured by producing the antiviral alginic acid conjugate fiber by mixing 5 to 10 wt % of sodium alginate polymer, 1 to 3 wt % of copper sulfate powder, and distilled water as a balance with each other and stirring them at 40 to 60° C. to produce a copper sulfate-alginate spinning solution, degassing the copper sulfate-alginate spinning solution in order to remove air bubbles, filtering the degassed copper sulfate-alginate spinning solution, quantitatively discharging the filtered copper sulfate-alginate spinning solution through a gear pump, coagulating the discharged copper sulfate-alginate spinning solution in a coagulation solution containing calcium chloride, sequentially passing the coagulation through a water tank and an oil tank, drying the coagulation, and then winding the coagulation. Here, copper chloride or other copper compounds may be used instead of the copper sulfate.

The caz fabric is a human-friendly fiber capable of maintaining an inactivation effect of the viruses by introducing copper ions known to be effective in inactivating the viruses into a spinning solution at the time of producing an alginic acid fiber and partially replace sodium ions in a sodium alginate polymer solution with the copper ions to allow copper ion particles to be carried in an alginate polymer and allow the copper ions to be uniformly distributed inside and outside the fiber and to significant reduce a possibility of separation of copper particles due to physical friction after the fiber is produced.

That is, the caz fabric is a conjugate fiber effectively inactivating a wide range of viruses such as coronaviruses such as severe acute respiratory syndrome (SARS) virus, a middle east respiratory syndrome (MERS) virus, and CVD10, a human influenza virus (H1N1 type), a bird flu virus (H5N1 type), and a CDV virus by spinning a conjugate fiber with a spinning solution in which copper ions in a copper sulfate solution and the sodium alginate polymer, which is an anionic polymer, are mixed with each other.

The caz fabric has an effect of killing 99.9% of the viruses within 5 minutes by an oligo dynamic action effect when it is in contact with the viruses.

Specifically, the caz fabric may be produced by the following method.

The copper sulfate-alginate spinning solution is produced by mixing 5 to 10 wt % of sodium alginate polymer, 1 to 3 wt % of copper sulphate powder, and distilled water as the balance and stirring at 40 to 60° C.

It has been found that Cu2+ ions are generally less effective than silver ions in terms of antibacterial properties but have an excellent effect in antiviral properties (Applied and environmental microbiology, April 2007, p. 2748-2750).

The present invention prepares a copper sulfate-alginate spinning solution by mixing copper sulfate as a salt having such copper ions with sodium alginate polymer. The copper sulfate is easily dissolved in an aqueous solution to form copper ions, which are partially replaced with sodium ions in the sodium alginate polymer solution to allow copper ion particles to be carried in the alginate polymer and to allow copper ions to be uniformly distributed inside and outside of the fiber. Also, since the binding of between the copper ions with sodium alginate polymer is not a simple physical binding, the possibility of detaching by the physical friction of the copper particles after the fiber is prepared is significantly reduced, and thus, it is also excellent in terms of persistence of the virus inactivation effect.

It is known that alginic acid used as the base material of the antiviral complex fiber in the present invention is extracted from brown algae, which is one of marine organisms, exhibits an acid property since it has a carboxyl group (COOH—) of uronic acid in the molecule, is usually used as sodium alginate (alginic acid sodium) in the form of sodium salt, and can be easily fiberized by spinning with a coagulation bath of a solution of calcium chloride (Encyclopedia of textile finishing, H. K. Rouette, Springer, 2000).

Alginic acid is non-toxic to human body and easy to process, and is used in the food, medicament and fiber industries because it is dissolved in water and exhibits high viscosity. Also, alginic acid is cross-linked with metal salts to induce gels, so it has recently attracted attention as a natural polymer material along with chitin, chitosan, etc., as wound dressing. In view of the chemical structure of alginic acid, as shown in the below figure of a structural isomer of the alginate polymer, alginic acid is a straight-chain copolymer in which a block of manuronic acid (M units), a block of gluronic acid (G units), and a block of MG units in the middle are linked by 1,4-glycoside bonds, and it is known that its physicochemical properties are influenced by physical properties such as viscosity, solubility, and ion-exchange ability due to differences in a M/G ratio, arrangement state of molecules, and molecular weight. In the present invention, the sodium alginate polymer in which a ratio of manuronic acid and gluronic acid (M/G ratio) is 0.6 to 1.2, is suitable for carrying the copper ions.

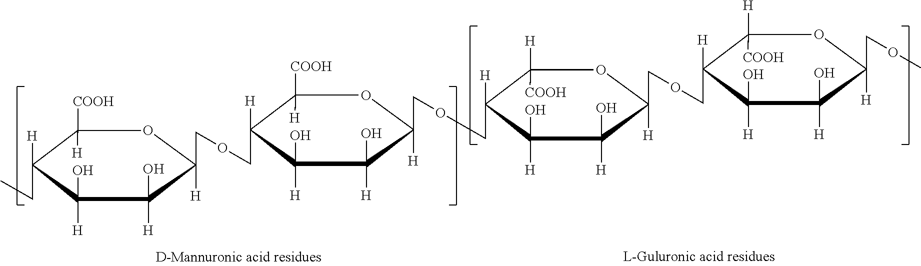

In the above figure, sodium alginate, which is prepared in the form of sodium salt (—COO—Na+) of a carboxyl group of alginic acid, is used as a carrier for a slow-releasing drug delivery system and as a carrier for proteins, enzymes, etc., and the carboxyl group which is an anion of sodium alginate, and the copper ion which is cation of sodium alginate, are easily bound through ionic bonding. In this case, sodium alginate preferably has a weight average molecular weight of 200,000 to 300,000, and a polydispersity index of less than 2.5.

The present invention is characterized in that a copper sulfate-alginate spinning solution in which 1 to 3 wt % of copper sulfate is mixed with the spinning solution is used, and if mixed with copper sulphate having copper ions in the spinning solution, it has the advantage of being able to quantify the content of the copper (12000 ppm=1.2%) which may represent antiviral effect. It has a disadvantage that the regulation of the content is difficult due to the outflow of copper components as in the conventional method, if the copper is added to the coagulation bath or to the process after the coagulation bath. Only if the content is less than 1 wt %, antiviral effect decreases, and if the content is very high more than 3 wt %, a gelation phenomenon caused by excessive substitution of copper ions and sodium ions occurs excessively, and the flowability of a spinning solution is reduced and the spinning workability is reduced.

Mixing about 12,000 ppm of nano-metallic copper particles in the spinning solution may be considered, but in this case, it is difficult to produce a uniform spinning solution due to the sinking of copper particles in the spinning solution, which causes nozzle clogging and yarn breakage during fiber spinning, making it virtually impossible to produce the fiber.

The copper sulfate-alginate spinning solution thus prepared is added into a spinning reservoir, depressurized to 0.5 torr to defoam the remaining air bubbles in the solution, and then air pressure is applied to quantify it with a gear pump to discharge the spinning solution quantitatively, through a hole formed in the spinning nozzle. Thereafter, the spinning solution is coagulated by sodium-calcium ion exchange reaction in a coagulation solution containing calcium chloride to form fibers. The spinning solution has a viscosity of 50,000 to 200,000 cps, and a concentration of 5 to 15 wt %, preferably 8 to 10 wt %.

The composition of the coagulating solution for coagulating sodium alginate is preferred that it consists of 5 to 10 wt % of calcium chloride, 30 to 70 wt % of ethanol and distilled water as the balance, and fibrosis is also achieved in a coagulation bath containing only calcium chloride, but it is preferable to mix 30 to 70 wt % of ethanol in the coagulation bath, thereby speeding up coagulation rate and at the same time minimizing the outflow of copper ions into the fiber.

Thereafter, in order to improve the physical properties through molecular chain orientation inside the fiber polymer, the fiber bundle is pulled at a draw ratio of 1.1 to 3.0 in hot water (40° C. to 70° C.), washed, softened, dried, and then wound. It is preferable that the copper content relative to the finally obtained fiber weight is 6000 to 12000 ppm (relative to the fiber weight).

In some cases, a post-spinning reduction process may be performed, but without the reduction process, Cu2+ ions are present inside and outside the antiviral complex fiber to maximize electrostatic attraction with the virus surface (phospholipid component) to maximize virus inactivation.

A heat-sealed non-woven fabric may be provided by mixing 5 to 50 wt % of the antiviral complex fiber thus prepared and 50 to 95 wt % of a low-melting polyester fiber or a bicomponent fiber (polyethylene/polypropylene).

In the following Examples, non-limiting examples of an antiviral alginic acid complex fiber of the present invention and a process of producing a non-woven fabric using the same are described.

### Example 1

10 wt % of sodium alginate polymer with a molecular weight of 200,000 and a polydispersity index of 2.5, 1.5 wt % of copper sulfate powder, and distilled water as the balance were mixed with each other and stirred at 40 to 60° C. to produce a copper sulfate-alginate spinning solution, and the copper sulfate-alginate spinning solution was defoamed under reduced pressure in order to remove air bubbles, filtered, quantitatively discharged through a gear pump, coagulated in a coagulation solution containing 10 wt % of calcium chloride, sequentially passed through a water tank and an oil tank, dried, and then wound. The copper content in the finally obtained alginic acid-copper fiber was about 6,000 ppm.

### Example 2

The complex fiber was produced in the same manner as in Example 1, except that the coagulating solution composition in Example 1 was used to be 8 wt % of calcium chloride, 50 wt % of ethanol, and distilled water as the balance. The copper content in the finally obtained alginic acid-copper fiber was about 12,000 ppm.

### Comparative Example 1

10 wt % of sodium alginate polymer with a molecular weight of 200,000 and a polydispersity index of 2.5, 1.2 wt % of nano-copper particles and distilled water as the balance were mixed with each other and stirred at 40 to 60° C. to produce a copper-alginate spinning solution, and the copper-alginate spinning solution was defoamed under reduced pressure in order to remove air bubbles, filtered, quantitatively discharged through a gear pump, coagulated in a coagulation solution containing 10 wt % of calcium chloride, sequentially passed through a water tank and an oil tank, dried, and then wound.

### Comparative Example 2

10 wt % of sodium alginate polymer having a molecular weight of 200,000 and a polydispersity index of 2.5 and distilled water as the balance were mixed with each other and stirred at 40 to 60° C. to produce an alginate spinning solution, and the alginate spinning solution was defoamed under reduced pressure in order to remove air bubbles, filtered, quantitatively discharged through a gear pump, coagulated in a coagulation solution containing 10 wt % of calcium chloride, 2 wt % of copper sulfate and distilled water as the balance, sequentially passed through a water tank and an oil tank, dried, and then wound.

### Comparative Example 3

The complex fiber was produced in the same manner as in Example 1, except that the spinning solution composition in Example 1 was used to be 10 wt % of sodium alginate polymer and 5 wt % of copper sulphate powder.

Table 1 shows the results of analyzing the copper content in the fiber of the thus-produced copper-alginic acid fiber by ICP. In addition, the virus inactivation effect of the produced fiber was measured using the EID50 (Egg Infective dose50) method, as follows. The produced fiber (sample fibrous materials), negative control (blank sample), and positive control were measured at 10 mg/ml in a 50 ml tube and added, mixed with 45 ml of an AIV virus solution, and then incubated in a 25° C. shake mixing incubator for 22 hours. 5 ml of the cultured sample was transferred to a new tube and centrifuged at 3,000 rpm for 30 minutes. After harvesting the supernatant, each 0.1 ml of the supernatant diluted in ten (10) fold was inoculated into the prepared SPF fertilized eggs, followed by incubation in a 37° C. incubator. After observation for 2 days, chilling was performed to harvest allantoic fluid, and a hemagglutination assay was performed. A hemagglutination assay (HA) is as follows. Virus diluted ½ step in PBS was dispensed in 50 μl increments in a 96 well micro plate, and an equal amount of 1% chicken red blood cells was dispensed. After standing at RT for 40 minutes, the results were read to confirm a HAU (Hemagglutination Unit) titer.

The virus inactivation efficacy of the fiber was calculated as LogEID50 value by using Equation as follows.

$$virus inactivation rate ( \% ) = \frac{( {10}^{{blank}^{ virus titer}} - 1 {0}^{virus titer of a sample} )}{( 1 {0}^{blank virus titer} )} \times 100 ( \% )$$

In addition, a comparison of the bacteriostatic reduction rate (KSK0693) for Staphylococcus aureus is made and the results are shown in Table 2.

In addition, the process of making a thin web (40 gsm or less in basis weight) is essential for use in a heat-sealed non-woven fabric (thermal bonding method), which is a commonly used as respiratory mask fabric, and requires relatively high fiber properties for strength and leveling. The fiber properties (strength and elongation) of Examples and Comparative Examples were measured and are shown in Table 3.

TABLE 1

| Items | Copper content (ppm) |
|---|---|
| Example 1 | 6,000 |
| Example 2 | 12,000 |
| Comp. Example 1 | Impossible fiber formation |
| Comp. Example 2 | 10,000 |
| Comp. Example 3 | Impossible fiber formation |

TABLE 2

| | Virus inactivation | Bacteriostatic reduction |
|---|---|---|
| Items | rate (%) | rate (%) |
| Example 1. | 90 | 80 |
| Example 2. | 99.9 | 90 |
| Comp. Example 1 | Impossible fiber formation | — |
| Comp. Example 2 | 92 | 85 |
| Comp. Example 3 | Impossible fiber formation | — |

TABLE 3

| Items | Strength (g/d) | Elongation (%) |
|---|---|---|
| Example 1. | 1.8 | 10 |
| Example 2. | 2.0 | 12 |
| Comp. Example 1 | Impossible fiber formation | — |
| Comp. Example 2 | 0.8 | 4 |
| Comp. Example 3 | Impossible fiber formation | — |

In the case of Comparative Example 1, the copper metal particles were precipitated in a solution and the nozzle was clogged during spinning, making fiber spinning impossible. In the case of Comparative Example 2, copper ions of copper sulfate in the coagulation bath inhibited the sodium alginate coagulation reaction of calcium ions in the calcium chloride solution, and physical properties of finally obtained fiber were very weak with a strength of 0.8 g/d and an elongation of 4%. In the case of Comparative Example 3, since the content of copper sulfate in a dope was too large, the gelation phenomenon of sodium alginate by copper ions occurred excessively, so that the flow of the solution disappeared, which made it impossible to prevent spinning.

It can be seen that the caz fabric formed by the producing method described above has a very high antibiosis (bacteriostatic reduction rate of 99.9%) against a Staphylococcus aureus as well as the virus, as illustrated in FIG. 4 (certified in antibacterial test (2020 Mar. 3) by KOTITI Research Institute). As illustrated in FIG. 5, the caz fabric has an effect of showing an inactivation rate of 99.8% or more in 1 minute (99.999% or more in 10 minutes) in an evaluation of inactivation of an indicator virus (MS-2 bacteriophage) (certified in virus sterilization test (2020 Mar. 16) by SELS Research Institute).

In this case, the virus killing portion 111 may be formed by spraying a nano-material forming a nano-membrane filter of the virus blocking portion 112 on the caz fabric (first producing method) or may be formed by spraying a nano-material forming a nano-membrane filter of the virus blocking portion 112 on a fabric, which is a smooth nonwoven fabric, and then overlapping the caz fabric with the nano-material (second producing method).

Here, in the first producing method, when the caz fabric is not smooth, there is a possibility that the filter will not be formed properly, and the virus killing portion 111 may be formed by the second producing method to eliminate this possibility.

Therefore, in the caz fabric produced by the producing methods as described above, copper ions in contact with the virus serve to destroy a shell protein of the virus through an oligodynamic action effect and at the same time decompose RNA of the virus to kill the virus, thereby providing an effect of sterilizing not only the virus but also a bacterium.

In addition, the virus killing portion 111 may be formed of a fiber having hydrophilicity. Because the virus (droplet) has a water-friendly property, that is, hydrophilicity, the virus killing portion 111 having such hydrophilicity may rapidly selectively adsorb and destroy the virus or a droplet containing the virus.

In addition, in another embodiment of the present invention, a filter portion 11 to be described above may be formed on an outer side of the suction portion 102, and the air sucked into the suction portion 102 may pass through the filter portion 11 and be introduced then into the air conditioning device body portion 10. Therefore, in another embodiment of the present invention, an air suction load is somewhat poor, but an effect of maximizing virus blocking efficiency is exhibited.

The suction portion 102 may include the filter portion 11 which is formed on the outer side thereof and in which the virus killing portion 111, the virus blocking portion 112, a first filter portion 113, and a second filter portion 114 are formed. Hereinafter, the filter portion 11 will be described in detail.

The filter portion 11 includes the virus killing portion 111, the virus blocking portion 112, the first filter portion 113, and the second filter portion 114.

The virus killing portion 111 will be replaced by the above description.

The virus blocking portion 112 is formed in a membrane form to block the virus.

The virus blocking portion 112 may have a form of a nano-fiber filter or have a form of a membrane filter.

In the virus blocking portion 112, a membrane filter is specially formed without disposing a charged melt blown filter (that is, an MB filter).

Referring to FIGS. 3A and 3B, the membrane filter has pores formed to be very smaller than those of the charged melt blown filter, such that even droplets of 1 μm or less may not pass through the membrane filter and are collected on a surface of the membrane filter. On the other hand, the charged melt blown filter has pores large enough for droplets of 5 μm or more to pass therethrough.

Usually, the charged melt blown filter (MB filter) uses a manner of sticking particles such as fine dust to the charged melt blow filter by an attractive force of static electricity, instead of having a very larger pore size than the membrane filter as illustrated in FIGS. 3A and 3B. Therefore, the charged melt blown filter (MB filter) has a larger pore size than the virus, such that the virus is blocked by the charged melt blown filter and passes through the charged melt blown filter at it is. In this case, the virus is stuck to an inner portion of the charged melt blown filter (MB filter) according to the attractive force of the static electricity, such that the virus remains alive in the charged melt blown filter (MB filter) for several hours or more.

In a case of applying such an charged melt blown filter (MB filter) together with the virus killing portion 111 according to the present invention, the virus is not stopped at the virus blocking portion 112 and passes directly through the virus blocking portion 112, such that the virus killing portion 111 may not exhibit an effect of secondarily destroying the virus.

Therefore, in the present invention, the virus killing portion 111 needs to be coupled to the virus blocking portion 112 on which the membrane filter is formed, rather than the charged melt blown filter, and furthermore, the virus killing portion 111 needs to be located in front of the virus blocking portion 112 rather than behind the virus blocking portion 112 in order to exhibit an effect of significantly improving an ability to kill the virus through the two-step virus destruction configuration as described above.

In addition, the virus blocking portion 112 may be formed of a membrane filter having hydrophobicity. Because the virus has a water-friendly property, that is, hydrophilicity, the virus blocking portion 112 having such hydrophobicity may repel the virus to the virus killing portion 111 having the hydrophilicity to help the virus to be easily adsorbed to the virus killing portion 111.

That is, the virus blocking portion 112 having the hydrophobicity blocks the virus or the droplet containing the virus that has passed through the virus killing portion 111 and repels the virus to the virus killing portion 111 having the hydrophilicity, such that virus killing efficiency of the virus killing portion 111 is maximized.

As described above, in the present invention, 1) both of the virus killing portion 111 and the virus blocking portion 112 are included in the air conditioning device 1, 2) the virus killing portion 111 is configured to be located in front of the virus blocking portion 112, and 3) the virus blocking portion is formed in the membrane form, such that the air conditioning device 1 may perform not only a function of blocking the virus but also a function of killing the virus to maximize safety of a user and efficiently prevent the spread of the virus.

The first filter portion 113 and the second filter portion 114 may filter foreign materials from the air sucked into the suction portion 102.

In this case, the filter portion 11 may be disposed on an outer side of the suction portion 102, and the virus killing portion 111, the virus blocking portion 112, the first filter portion 113, and the second filter portion 114 of the filter portion 11 may be formed so that the virus killing portion 111 is formed at the forefront of the suction portion 102 and the virus blocking portion 112, the first filter portion 113, and the second filter portion 114 are sequentially disposed toward a rear.

In summary, the air conditioning device 1 according to an embodiment of the present invention includes the virus killing portion 111 disposed in front of the suction portion 102 and having the differential pressure exceeding 0 Pa and less than 10 Pa to be capable of killing the virus in the sucked air without substantially hindering the air suction of the air conditioning device 1.

In addition, the air conditioning device 1 according to an embodiment of the present invention may include both of the virus killing portion 111 and the virus blocking portion 112 to perform not only the function of blocking the virus but also the function of killing the virus, thereby maximizing the safety of the user and efficiently preventing the spread of the virus.

In addition, in the air conditioning device 1 according to an embodiment of the present invention, the virus killing portion 111 is configured to be located in front of the virus blocking portion 112, such that the viruses are primarily destroyed while passing through the virus killing portion 111, and the residual viruses that are not destroyed by the virus killing portion 111 and pass through the virus killing portion 111 are blocked by the virus blocking portion 112 to be collected on the surface of the virus blocking portion 112 and are again secondarily destroyed by the virus killing portion 111. As a result, an ability to kill the virus may be significantly improved.

In addition, in the air conditioning device 1 according to an embodiment of the present invention, the virus killing portion 111 is produced using the caz fabric, such that an antibacterial ability is improved as compared with an existing antibacterial fiber.

Although the present invention has been described in detail through specific embodiments, these embodiments are to specifically describe the present invention, the present invention is not limited to these embodiments, and it will be obvious that various modifications and alterations may be made by those skilled in the art without departing from the scope and spirit of the present invention.

All of simple modifications and alterations of the present invention will be considered to fall within the scope of the invention, and a specific protective scope of the invention will become clear by the claims.

1. An air conditioning device that sucks air through a suction portion and sending the sucked air to a blowing portion to perform air conditioning,
wherein the suction portion includes a virus killing portion formed of a fiber to which copper ions are bonded and killing a virus,
the virus killing portion includes a caz fabric configured to contain 5 to 50 wt % of an antiviral alginic acid conjugate fiber configured so that the copper ions are contained in an amount of 6,000 to 100,000 ppm in a fiber, and
the virus killing portion has a differential pressure exceeding 0 Pa and less than 10 Pa.

2. The air conditioning device of claim 1, wherein the caz fabric is configured by producing the antiviral alginic acid conjugate fiber by mixing 5 to 10 wt % of sodium alginate polymer, 1 to 3 wt % of copper sulfate powder or copper chloride powder, and distilled water as a balance with each other and stirring them at 40 to 60° C. to produce a copper sulfate-alginate spinning solution, degassing the copper sulfate-alginate spinning solution in order to remove air bubbles, filtering the degassed copper sulfate-alginate spinning solution, quantitatively discharging the filtered copper sulfate-alginate spinning solution through a gear pump, coagulating the discharged copper sulfate-alginate spinning solution in a coagulation solution containing calcium chloride, sequentially passing the coagulation through a water tank and an oil tank, drying the coagulation, and then winding the coagulation.

3. The air conditioning device of claim 2, wherein the suction portion further includes a virus blocking portion formed in a membrane form to block the virus or a droplet containing the virus, and
the virus killing portion is configured to
primarily destroy the viruses passing through the virus killing portion, and
be located in front of the virus blocking portion so as to secondarily eradicate residual viruses collected on a surface of the virus blocking portion by the virus blocking portion when the virus blocking portion blocks the viruses from passing therethrough, such that the viruses are collected on the surface of the virus blocking portion.

4. The air conditioning device of claim 3, wherein the virus killing portion is formed of a fiber having hydrophilicity, and
the virus blocking portion is formed of a membrane filter having hydrophobicity.

5. The air conditioning device of claim 3, wherein the virus blocking portion has a form of a nano-membrane filter, and
the virus killing portion is formed by spinning a nano-fiber forming the nano-membrane filter of the virus blocking portion on a fabric and then overlapping the caz fabric with the nano-fiber to be configured to be located in front of the virus blocking portion.

6. The air conditioning device of claim 5, wherein the fabric is a smooth nonwoven fabric.

7. The air conditioning device of claim 3, wherein the virus blocking portion has a form of a nano-membrane filter, and
the virus killing portion is formed by spinning a nano-fiber forming the nano-membrane filter of the virus blocking portion on the caz fabric in an electro-spinning manner to be configured to be located in front of the virus blocking portion.

8. The air conditioning device of claim 3, wherein the suction portion further includes a filter portion filtering foreign materials sucked in the air sucked through the suction portion, and
the suction portion is formed so that the virus killing portion is formed at the forefront of the suction portion and the virus blocking portion and the filter portion are sequentially toward a rear.

9. The air conditioning device of claim 1, wherein the air conditioning device is at least one of an air conditioner for an interior, an air conditioner for a vehicle, an air cleaner, an electric fan, or a heater.
